(19) United States
(12) Patent Application Publication
(10) Pub. No.: US 2008/0003265 A1
(43) Pub. Date: Jan. 3, 2008

(54) Protein and fiber containing dietary supplement

(76) Inventors: John Francis Casey, Summerfield, FL (US); Hans Fisher, Highland Park, NJ (US)

Correspondence Address: Herbert W. Mylius, 9139 SE 120th Loop, Summerfield, FL, 34491, US

(21) Appl. No.: 11/476,483

(22) Filed: Jun. 28, 2006

(51) Int. Cl. A61K 47/00

(52) U.S. Cl. 424/439

(57) ABSTRACT

A dietary supplement, in powder, capsule, pill, or packet form, suitable for weight reduction as well as for cholesterol lowering and general colon health, comprises a soy protein component in addition to a plurality of fiber components selected from the group consisting of soy fiber, oat fiber, psyllium, sesame flour, flaxseed, barley, indigestible dextrin, beet fiber, rice bran, guar gum, pectin, and carageenan.

### CROSS REFERENCE TO RELATED APPLICATIONS

None.

### FEDERALLY SPONSORED RESEARCH AND/OR DEVELOPMENT

None.

### BACKGROUND OF THE INVENTION

1. Field of the Invention

Diet and dietary supplements have become a major focus of research into overall health, in light of the high and growing incidence of obesity, particularly in youth and children. Other areas of major concern include high blood cholesterol, which leads to heart disease, the number one cause of death; early onset diabetes; and increasing cancer rates, including colon and other cancers possibly traceable to poor dietary habits. The present invention is directed to an improved dietary supplement comprising a plurality of specific fibers in combination with a specific protein source, which unique combination provides benefits both in weight control, digestive health, and lowered blood cholesterol.

2. Background Art

It is well established that high fiber diets are conducive to improved digestive health, as well as to the control of weight and cholesterol. It is known that the typical American diet must share in the blame for the high incidence of obesity in this country. It is further known that increased bulk, resulting from an increase in fiber ingestion, contributes to better Taxation, aiding food and the end products of food digestion to pass through the digestive tract without dwelling and accumulating in sections of the lower colon, where bacteria may cause the production of toxic substances which lead to inflammation such as diverticulitis, or much worse, cancer of the colon. Colon cancer takes the lives of more than 28,000 men each year, and nearly as many women, placing it second only to lung cancer among fatal cancers. New studies show that dietary fiber, previously thought to not aid against colon cancer, has a large role in the prevention thereof. By reducing the dwell time of digesta in the colon, one reduces the development of toxins, contributing appreciably to the general well-being and health of the colon. Fiber also can produce a lowering of blood cholesterol by binding cholesterol and fats in the digestive tract and preventing them from being absorbed. Still further, it is known that certain types of soluble and insoluble fiber are beneficial in the prevention and treatment of diabetes and heart disease.

As indicated, the use of diet supplements to improve the degree to which fiber and other beneficial elements are ingested is a well-known element of nutrition. While it is also known that many forms of fiber may be employed in such diet supplements, no teachings have been found which employ a combination of multiple fiber components, as specified in the present invention, and specifically combine a plurality of fibers with protein to achieve a synergistic benefit upon overall health. In general, those references that teach the use of dietary fiber as diet supplements avoid the addition of protein to the diet supplement, and use only a single fiber to provide bulk to aid in avoidance of constipation.

For example, there are many references to the use of dietary fiber in general for such benefits as cholesterol control, Taxation, and relief of constipation. In addition, a recent posting on the Mayo Clinic website, Mayoclinic.com/healthtfiber, entitled “Dietary fiber: An essential part of a healthy diet” by the Mayo Foundation for Medical Education and Research, indicates that dietary fiber, best known for its ability to prevent or relieve constipation, can also lower the risk of diabetes and heart disease. Dietary fiber, also known as roughage or bulk, is commonly understood to include all parts of plant foods that the body can't digest or absorb. Fiber is often classified into two categories: that which doesn't dissolve in water (insoluble fiber) and that which does (soluble fiber). The insoluble fiber is beneficial and necessary for movement of material through the digestive system, while the soluble fiber helps lower blood cholesterol and glucose levels. It is recommended that one include a wide variety of high fiber foods in ones diet to obtain the greatest health benefit.

In addition, Li et al, in “Effects of fiber intake on the blood pressure, lipids, and heart rate in Goto Kazizaki rats”, Nutrition, 2004 Nov-Dec; 20 (11-12): 1003-7, conclude that high fiber intake has beneficial effects on systolic blood pressure and blood lipid levels, and that fiber intake should be increased by individuals who have diabetes mellitus.

Further, many studies of the effect of specific fiber sources on health have been published, including the following. Panlasigui et al, in “Blood cholesterol and lipid-lowering effects of carageenan on human volunteers”, Asia Pac J Clin Nutr. 2003;12(2):209-14 indicate that the regular inclusion of carageenan in the diet may result in reduced blood cholesterol and lipid levels in human subjects. Behall et al, in “Comparison of Hormone and Glucose Responses of Overweight Women to Barley and Oats”, J Am Coll Nutr. June 2005; 24(3); 182-8, show both oat and barley meals reduced glycemic responses; the high soluble fiber content of the barley appeared to be a factor in the greater reduction observed. The same authors, in an earlier published article, “Lipids Significantly Reduced by Diets Containing Barley in Moderately Hypercholesterolemic Men”, J Am Coll Nutr. Febuary 2004; 23(1): 55-62, concluded that increasing soluble fiber through consumption of barley in a health diet can reduce cardiovascular risk factors. In “Reduced glycemic response to beet-fibre meal in non-insulin-dependent diabetics and its relation to plasma levels of pancreatic and gastrointestinal hormones,” Diabetes Res. Febuary 1986; 3(2): 91-6, Hagander et al observe that diminished glycemic response after beet-fiber ingestion is associated with an increased response of somatostatin, giving a reduced glucose absorption and a delayed gastrointestinal transit time. Similarly, Overton et al, in “The effects of dietary sugar-beet fibre and guar gum on lipid metabolism in Wistar rats,” Br J Nutr. September 1994; 72(3):385-95, indicate that guar exerts its hypocholesterolemic effect via intraluminal bile acid binding and loss of cholesterol from increased fecal bile acid excretion. Oat bran is discussed in “Oat-bran cereal lowers serum total and LDL cholesterol in hypercholesterolemic men”, Am J Clin Nutr. September 1990; 52(3): 495-9, where Anderson et al conclude that ready-to-eat oat bran cereal provides a practical means to incorporate soluble fiber into the diet to lower serum cholesterol. In “Long-term cholesterol-lowering effects of psyllium as an adjunct to diet therapy in the treatment of hypercholesterolemia”, Am J Clin Nutr. June 2000; 71(6): 1433-8, Anderson et al teach that treatment with 5.1 g psyllium twice daily produces significant net reduction in serum total and LDL-cholesterol concentrations in men and women with primary hypercholesterolemia, providing an alternative to drug therapy for some patients. Rice bran is discussed in “Full-Fat Rice Bran and Oat Bran Similarly Reduce Hypercholesterolemia in Humans”, J Nutr. May 1998; 128(5): 865-9, by Gerhardt et al. Sesame is similarly discussed in “Sesamin, a Sesame Lignin, is a Potent Inducer of Hepatic Fatty Acid Oxidation in the Rat”, Metabolism. October 1999; 48(10): 1303-13, by Ashakumary et al.

Of course, protein is a wellestablished necessity in the human diet, and may be obtained in a number of forms. Sources of animal protein include meats, egg albumin, whey, and caseinates. Other sources of protein include plant material, such as sunflower, linseed, safflower, peanut, almond, walnut, cottonseed, and sesame seed. The preferred protein for use in the present invention is, however, soy protein, which is readily available, and a known ingredient of food supplements and dietary supplements. However, as previously stated, no previous food supplements have employed soy protein in combination with a plurality of fiber sources as set forth herein. Even those diet supplements designed specifically for weight loss, which commonly employ a protein source, fail to recognize or utilize the advantage of multiple fibers in combination with protein in their compositions.

The benefit of soy protein in the diet rather than animal protein is discussed by Anderson et al, in “Meta-analysis of the Effects of Soy Protein Intake on Serum Lipids” N Engl J Med. August 1995 3;333(5):276-82, in which it is concluded that the consumption of soy protein rather than animal protein significantly decreased serum concentrations of total cholesterol, LDL cholesterol, and triglycerides without significantly affecting serum HDL cholesterol concentrations. Carroll, in “Review of clinical studies on cholesterol-lowering response to soy protein”, J Am Diet Assoc. July 1991; 91(7):820-7, relates that soybean protein has hypocholesterolemic and anti-atherogenic properties.

### BRIEF SUMMARY OF THE INVENTION

The present invention relates to ingestible dietary supplements for the purpose of providing appropriate nutritional elements to the human diet. More particularly, this invention relates to a unique composition comprising a specific combination of both protein and a plurality of dietary fibers. The specific selection of the components of the inventive composition is based upon obtaining an optimal choice and concentration of amino acid profiles in the protein component, and the best balance of fibers for overall effect. The fibers are chosen so as to obtain an optimal balance of the most effective percentages of soluble fiber, for influencing cholesterol levels, and soluble, semi-soluble or insoluble fiber for overall colon health. For example sesame seed and soy proteins complement each other in so far as their respective amino acid profiles are concerned. While soy is high in lysine, sesame is low in lysine. Conversely, sesame's higher level of tryptophan compensates for the lower level of this amino acid in soy protein.

### DETAILED DESCRIPTION OF THE INVENTION

The present invention is directed to an ingestible food supplement so formulated as to have the maximum beneficial results. First, the formulation is designed to provide a mixture of fibers in combination with protein in a convenient form to be ingested shortly prior to or as part of a meal, which will produce early satiety in one consuming the product, and thereby enable the individual to reduce his or her food intake during the meal. A further beneficial effect of the composition is to prevent the absorption of dietary cholesterol and fats from the meal, thereby denying the liver the fats that serve as precursors of cholesterol. Still further, the dietary fibers employed are believed to be beneficial in the possible prevention of colon cancer, and in reducing the risk of diabetes, intestinal problems, and heart disease. Moreover, by combining these fibers with the specified protein employed in the present invention, one obtains an optimal combination of protein and amino acid profiles for overall health.

Satiety is dependent upon two principal physiological needs that are best satisfied simultaneously. The first of these is the need for caloric intake; the second is the need for bulk. If one provides a high density, high calorie diet, as for example with a low fiber, high fat diet, one inevitably tends to overeat beyond the need for calories in order to satisfy the need for bulk. It is believed that many Americans become obese because of the tendency to follow this dietary trend of eating foods low in fiber and high in fat. It is, of course, also possible to eat a diet so high in fiber or bulk as to prevent the ingestion of sufficient calories to meet the caloric needs, although this seldom happens in Western society. Under those conditions, the person will eventually suffer from malnutrition. The importance of bulk in conjunction with adequate caloric intake results from the fact that digestion is facilitated through the activation of nerve endings in the walls of the intestine, brought about through pressure from the inside of the intestine resulting from the volume of food mixed with moisture. Thus, the more fiber in the diet the more readily the volume will increase and produce pressure against the intestinal walls, which triggers nerve endings to cause movement of the digestive tract. This movement, referred to as peristalsis, is responsible for the propulsion of the food through the digestive system, leading to the completion of the digestive process and the absorption of the nutrients. More-over, fiber, through its bulkiness, contributes to better laxation which means that food and the end products of food digestion pass through the digestive tract without accumulating in sections of the lower bowel, where bacteria can act on them and produce harmful toxins that can cause inflammation such as diverticulitis, or much worse, cancer of the colon.

It is well known that dietary fibers are considered an essential part of a healthy diet, providing many health benefits. The term dietary fiber is a generic term given to indigestible fractions in foods, which human digestive enzymes cannot digest. Dietary fibers, now considered an important aspect of diet, are found in fruits, vegetables, whole grains, and legumes, and are probably best known for their ability to prevent or relieve constipation. But dietary fibers can provide other important health benefits as well, such as contributing to the overall health of the colon, and lowering one's risk of diabetes and heart disease. As set forth in the present invention, we have found it beneficial to provide a mixture of soluble dietary fiber and insoluble dietary fiber to achieve an optimal mix of fibers to be ingested. Dietary fibers are believed to have a number of health benefits, perhaps the best known of which is to lower serum cholesterol level. This effect, however, is not uniformly found for all types of dietary fibers, due to different physiological properties and differing physicochemical properties, dependent upon the sources of the dietary fibers. Certain types of fiber bind dietary cholesterol in the digestive tract, thus preventing it from being absorbed, while others bind bile salts. Many studies have shown other benefits of fiber as well, such as reduction of the risk of diabetes. In this disease, fibers such as guar gum, carageenan, or pectin bring about a much slower rate of sugar absorption, thus producing a much smaller stimulation of insulin secretion, thereby helping the diabetic patient. It is also believed that intestinal problems, such as the risk of diverticulitis, a condition suffered by half of Americans over the age of 60, can be substantially reduced by the consumption of a high-fiber diet. Moreover, a recent Harvard study, for example, found that over a six-year period, the men who ate the most fiber (averaging 28.9 grams a day) had 41% fewer heart attacks compared with the men who ate the least. Dietary fiber is generally obtained from plant foods, and consists of that portion of the plant not digested by man. While the sugars and starches are broken down into simpler nutrients and are absorbed by our intestine, the cell walls are not digested and go on to form an important component of the stool. An example of dietary fiber is cellulose, and a food high in fiber is wheat bran. Dietary fiber, also referred to as roughage or bulk, includes all parts of plant foods that the body can't digest or absorb. Fiber is often classified into two categories: those that don't dissolve in water (insoluble fiber) and those that do (soluble fiber). Insoluble fiber increases the movement of material through the digestive system and increases stool bulk, so it can be of benefit to those who struggle with constipation or irregular stools. Whole-wheat flour, wheat bran, nuts and many vegetables are good sources of insoluble fiber. Soluble fiber dissolves in water to form a gel-like material. It can help lower blood cholesterol and glucose levels. Generous quantities of soluble fiber are found in oats, peas, beans, apples, citrus fruits, carrots, barley and psyllium.

Thus, there is much to be gained by ingesting dietary fiber, but to get the most benefit, it is important to provide a good mix of high-fiber foods. Fiber is found in abundance in vegetable products such as the bran of whole grains, the leaves and stems of plants, and nuts, seeds, fruits, and vegetables. However, the normal diet of most adults is deficient is some or all of these products. Also, it is known that recommended daily dosages for fiber are often relatively high, requiring the patient to ingest the fiber composition several times daily. Patient compliance is also a problem when the composition has an unpleasant taste, after-taste, or gritty mouth feel. Accordingly, a goal of the present invention is to provide to the consumer an easily and readily ingested product that will provide a proper balance of the best fibers, and additional protein, to attain the aforementioned benefits, and at the same time provide a tool to control weight and cholesterol. The addition of fiber to the diet for weight control is known, as fiber generally provides few if any calories to the diet, while providing satiety. However, it is not generally recognized by the public that low calorie diets may result in a breakdown of protein in the body, particularly from the digestive tract, and a corresponding loss of potassium associated with cell nucleus protein loss. Potassium loss is known to result in cardiac issues, as well as general health problems. Accordingly, to protect the digestive tract and to prevent possible protein breakdown, we have found it appropriate to provide a source of protein in combination with the fibers provided in the present dietary supplement.

To achieve an optimal product, it has been determined that a mixture of up to about ten specific fiber sources should be combined with a fortified protein source. While alternative protein sources are available, soy protein is considered the best protein for the present invention. It is to be noted that various forms of soy protein are available. For example, soy flour comprises about 50 percent protein, while soy concentrate comprises from about 70 to 72 percent protein, and soy isolate comprises from about 85 to 92 percent protein. Such sources of soy protein are commonly extracted using the solvent hexane, but the preferred soy isolate for use in the present invention is extracted with water and ultra filtration. Of course, the preferred fortified soy isolate protein, in combination with fewer than ten fibers will provide a benefit as well, although the maximum benefit is achieved through the use at least four, and preferably six, of the fiber components, each of which adds a specific benefit to the mixture of components. While the present composition will provide beneficial results with as few as one or two fibers, far superior results are obtained through the use of at least four fibers. However, as one adds additional fiber components, less of each, and thus less of the specific benefit of each individual fiber. component, is attained.

The food supplement of the present invention takes into consideration the fact that obesity and blood cholesterol are inextricably interwoven, and the present invention comprises a meal supplement concentrate, rather than a meal replacement. Intended to aid in both weight and cholesterol control, it is formulated with a plurality of natural ingredients, each available from a leading food processor. The optimal product is produced without the use of solvents or additives of any kind, is a fine granulate, and may be conveniently packaged in pill or capsule form, or in packets or pouches, for individual use prior to a meal as an addition to a liquid carrier, or, alternatively, may be added to a food product as a supplement thereto in the preparation thereof. Further, the product may be made available as an emulsion for addition to liquid containing foods, and may be flavored or neutral. As a fine granulate, the product is readily dispersible in any liquid, hot or cold, or can be used in cooking or baking without compromising effectiveness, or may be provided as small flavored nuggets (from 2 to 3.5 mm in diameter, for example) for various applications such as cereals (preferably hot), nutrition bars, and confectionary items. The product may be produced for consumption with water, juices, sport drinks, applesauce, sauces, salad dressings, gravies, puddings, yogurt, etc, at the users option, as well as an additive to omelets, hamburger patties, hamburger helper, or other foods, or may be baked into or included in bread, rolls, cookies, pretzels, meat loaf, pancakes, or similar products. The preferred formulation was developed with such applications and uses in mind, and thus to conform or harmonize with such applications. The present invention is specifically directed to a dietary supplement preferably in the form of pills, capsules, or packets, to be taken a specific number of times daily for the desired benefit.

The product comprises about 26 to 36 percent (by weight) soy protein, preferably in the form of soy protein isolate, the balance comprising a plurality of fibers, with up to about 2 percent flavoring and/or coloring agents. It is also to be noted that there are significant amounts of protein in some of the fiber components utilized. Specifically, there are protein components present in psyllium, soy fiber, sesame, and flax, which are additive to the soy protein isolate. The preferred harmonious blend of soy protein and up to about 10 specific fibers, with soluble and insoluble fractions, was based upon a careful consideration of the amino acid profile and the physiological characteristics of the ingredients, to achieve the greatest possible degree of success in cholesterol reduction and overall colon health. However, the synergistic effect of the combination of fibers suggested is not sufficient in and of itself, and the user should follow a recommended regimen of consuming at least 4 glasses of water daily, and slowly introduce moderate improvements in dietary practices. The use of three doses daily of the present invention, prior to meals, is beneficial to those marginally overweight or entering into levels of serum cholesterol above normal, but is not intended for those who are pathologically obese or suffering from hyperlipidemia. The proposed daily dosage of about 30 grams, when combined with the fiber in an average diet, should be ideal for most normal consumers, enabling them to reap the benefits of a realistic and moderate program that will lead them progressively toward modest weight loss and maintenance, cholesterol control, and general colon health.

Based upon a recommended usage of 30 grams per day of the dietary supplement, to be taken before each meal in individual servings of 10 grams, the product may be conveniently provided in the form of pills, capsules, or tablets, or may be packaged in 10-gram packets of powder. It is to be noted that the invention is not intended to replace any meals, but as a meal supplement, and the consumer is encouraged to consume a normal dietary regimen of three meals daily. The packets may be any of those known in the art as being suitable for food grade packaging, and may be heat-sealed to protect the product. Shelf life of the product is highly extended, as it contains less than 5 percent moisture. The individual serving packets may be packaged in larger packages for convenience and sale to the consumer, with informational and instructional materials included as appropriate, or readily available to the consumer through an internet web site, or by other means. The pills, capsules, or tablets may be prepared in conventional manner, and may be packaged in conventional packaging for the convenience of the consumer. For example, individual 10-gram capsules may be provided in blister packs of three for daily dosage, with multiple packs provided in a package, for example, of 30, or 90, for marketing purposes.

The present invention comprises protein, selected from the group consisting of soy, whey, caseinates, safflower, sunflower, linseed, almond, peanut, walnut, cottonseed, and sesame proteins, and preferably in the form of soy isolate protein, and a plurality of fibers selected from the group consisting of soy fiber, oat fiber, psyllium, sesame flour, flaxseed, barley, indigestible dextrin fiber, beet fiber, rice bran, guar gum, pectin, and carageenan. The individual components of the mixture are provided in dry particulate form. Since the particle sizes of the ingredients vary, after careful blending of the ingredients, the first step is to provide a fairly uniform granular size, through physical shear. Particles are preferably micro cut to facilitate dispersion in most foods to which it may be added. No effort need be made to obtain complete uniformity, but merely to bring the particles within a harmonious size range to allow uniform mixture and easy dispersion, as well as good mouth feel. The preferred particle size distribution of the product is such that about 10 percent of the product shall be greater than 850 microns, about 40 percent shall be from about 425 to about 850 microns, about 36 percent shall be from about 150 to about 425 microns, about 5 percent from about 75 to about 150 microns, and about 9 percent less than 75 microns. It is to be noted that, preferably, no solvents or additives are utilized in the manufacture of the inventive product, and particularly for the protein component thereof. It has been found that the use of solvents appears to deteriorate the protein component of the mixture, resulting in a decrease in bioavailability and efficacy. However, hexane solvent extraction, while not preferred, is generally recognized as safe, and may be used.

As indicated, the optimal product comprises a mixture of from four to about ten, and preferably about six or seven, specific forms of fiber, plus a fortified soy protein isolate. However, positive results are found to result from the use of a mixture of the soy protein source and one or more members of the group consisting of soy fiber, oat fiber, psyllium, sesame flour, flaxseed, barley, indigestible dextrin fiber, beet fiber, rice bran, guar gum, pectin, and carageenan. While from one to three fibers will provide a beneficial result, more improved results are, of course, achieved when at least four fiber sources are utilized, and, preferably, both soluble and insoluble fibers should be used. If more than about six or seven fibers are used, the quantity of each of the fibers is decreased, resulting in a decrease of the effect of each. The four preferred fibers for use are oat, psyllium, guar, and soy fibers. To achieve the maximum benefit, it is preferred that there be more insoluble fiber in the supplement than soluble fiber. The preferred amount of soluble fiber should be about 30 percent by weight of the total fiber. It is also to be noted that this mixture of ingredients is free of gluten, which fact would be of important therapeutic value to those suffering gluten intolerance, i.e. celiac disease, and the need to avoid wheat fibers. The present invention, being gluten free, may be added to hamburger, omelets, soup, etc., to improve the nutritional balance of those on gluten free diets by providing additional protein to their diet.

The villi, hair like strings found in the intestinal tract, function to draw nutrients from digested food. It is known that the villi are often damaged in patients who are frequently constipated, suffer from celiac, Krohn's, or other intestinal diseases, frequently use harsh laxatives, or have poor diets. By damaging the efficiency of the villi, one opens the door to debilitating diseases and/or affecting one's general health and well-being.

The invention, when properly administered, fosters improved laxation, and faster movement of material through the intestines without harming the villi. In addition, the specific group of protein and fibers chosen for inclusion in the present invention are believed to provide an optimal balance of soluble and insoluble fibers for colon health, as well as providing an ideal pattern of amino acids and types of protein for overall health and well-being. Insoluble fibers form the bulk needed to stimulate the movement of the intestines during digestion. This movement, peristalsis, helps propel food and the products of digestion from one end of the intestines to the other, in a wave-like motion that does not necessitate that the intestines alter their location in the abdominal cavity. The insoluble fibers, or any bulk, i.e. food even without fiber, stimulate nerve endings in the wall of the intestines, and these in turn set up a wave-like motion. If the food is low in fiber, larger amounts of food, and thus more calories, are needed to bring about the peristaltic motion. The more fiber, the fewer the calories required. Also, the more motion, the less chance of food and digesta lodging in the turns of the intestines, where bacteria can produce toxic or carcinogenic compounds that may give rise to cancer (of the colon). Soluble fiber also produces bulk, but, in addition, because of its water solubility can trap certain substances, particularly saturated fatty acids and cholesterol, which will be embedded within the soluble fiber and excreted in the feces.

As previously indicated, many fibers have been found to provide the necessary properties desired for the present invention. Positive results are obtained when the fiber component of the invention comprises preferably four or more fibers of the group consisting of soy fiber, oat fiber, psyllium, sesame flour, flaxseed, barley, indigestible dextrin fiber, beet fiber, rice bran, guar gum, pectin, and carageenan, although optimal benefits result from about six or seven of the members of the group.

The soy fiber component of the composition has attributes similar to rice, flax, and oat fibers, while having additional components present which have not as yet been fully explored or identified. Suffice it to say that soy fiber, or soy flour, has been shown to be effective as a bulk forming agent as well as a cholesterol-lowering type of fiber. A preferred soy fiber is available from The Solae Company, of St. Louis, Mo., as Fibrim™ 1020.

Oat bran is a well-known source of oat fiber, long recognized as having beneficial cholesterol-lowering properties. Dutch nutritionists were so impressed with these properties that oat meal-containing breads became a standard in The Netherlands almost 20 years ago. Oat bran is a particularly effective oat fraction currently being utilized in connection with cholesterol-lowering regimens, and is available in countless different formats, from baked goods to breakfast cereals. A preferred oat bran is commercially available from Canadian Harvest, of Cambridge, Minn., under the designation Oat Fiber 300.

Psyllium hulls, or husks, have been used for many years as part of such common laxatives as Metamucil®. Its beneficial properties as a laxative relate to its bulk-forming characteristics in the presence of an adequate intake of water. More recently, it has been shown that psyllium can reduce blood cholesterol in a similar fashion as do some of the other fibers that have already been discussed. From the scientific literature, it appears that psyllium acts by a different mechanism from that of other fibers. A suitable source of psyllium is Frutarom Meer Corp. of North Bergen, N.J.

Sesame flour is a light-brown powder obtained from oil-extracted sesame seeds. Sesame flour not only has high protein content, it also has an excellent amino acid profile, and shows good emulsifying activity and stability. Sesame flour is available from Dipasa USA, of San Antonio, Tex.

Flaxseed is considered to be heart-healthy because it contains lignans, which have antioxidant properties, and also contains omega-3 fatty acids, which aid blood pressure regulation, and favorably affect blood clotting, possibly lessening the chance of a fatal heart attack. Since the fiber in flaxseed is soluble (similar to oat fiber) it is also helpful in managing cholesterol levels. A suitable flaxseed is available as BevGrad™ whole grain milled flaxseed from Pizzey's Milling of Gurnee, Ill.

Barley is a well known form of fiber. It was noted in the late 1960's that spent barley hulls left over from the malting process were particularly efficacious in connection with the lowering of cholesterol. A number of laboratories, including those of the United Stated Department of Agriculture, have confirmed early findings, and barley fiber is a proven, excellent addition to the cholesterol lowering fibers now available for human consumption. A preferred form of barley fiber is available as Perlene Barley Flour, from Mid America Food, of Northbrook, Ill.

Indigestible dextrin is a kind of water-soluble dietary fiber, which has been reported to improve bowel movements and fecal conditions of healthy adults. A number of studies, specifically in Japan, have evaluated indigestible dextrin as both a pure foodstuff, and the effects of indigestible dextrin in processed foods. A suitable indigestible dextrin material is produced by a combination of heat and enzymatic treatment of cornstarch, as detailed in U.S. Pat. No. 5,620,873, issued Apr. 15, 1997, to Ohkuma et al, and U.S. Pat. No. 5,358,729, issued Oct. 25, 1994, to Ohkuma et al. A preferred source of indigestible dextrin fiber is Fibersol-2™ digestion resistant maltodextrin, a highly soluble fiber, available from ADM Corporation of Decatur, Ill. An alternative source of indigestible dextrin fiber is Eridex™, a bulk sweetener derivative of erithritol available from Cargill, Inc., of Minneapolis, Minn.

Some years ago, sugar beet pulp was an important commercial source of pectin. The importance of beet fiber as a fiber source in the human diet was accidentally determined when dried sugar beet pulp was added to an animal diet high in foods that would normally cause an increase in blood cholesterol. To the surprise of the investigators, the sugar beet pulp prevented the anticipated rise in blood cholesterol. Moreover, a discovery by German researchers showed that the addition of beet fiber in bread dough helps the bread to stay fresh longer, by binding water in the dough. In addition, it is believed that beet fiber contains components, other than pectin per se, which may also contribute to bulk formation as well as lowering cholesterol. Beet fiber has also been shown to reduce the blood sugar level in diabetics and to be beneficial to the gastrointestinal system. A suitable beet fiber is available as Fibrex Beet Fiber, from International Fiber Corp. of North Tonawanda, N.Y.

Guar gum has been used in small amounts as a food additive for many years because of the properties it contributes in connection with texture, mouth feel and appearance of the food to which it has been added. Guar gum is one of the most efficient of the cholesterol-lowering fibers. Furthermore, guar gum has been most advantageously used for certain diabetic patients to make them less dependent on insulin. However, one note of caution must be observed when formulating with guar gum, since guar has a tendency to increase laxation, when used too liberally. Guar gum is available from a number of sources, such as TIC Gums, of Belcamp Md., as Guar NT 3500 F.

As previously indicated, sugar beet pulp was an important commercial source of pectin. This source was later completely replaced by citrus and apple peel as the major source of pectin throughout the world. The efficacy of pectin as a cholesterol-lowering agent was determined some time ago. In the present invention, in addition to or in place of beet fiber, a less concentrated form of pectin may be used, which provides all of the beneficial effects thereof without some of the disadvantages of pure pectin, which can cause gumminess and stickiness in a dietary supplement. A suitable pectin is Pectin 1694, a product of TIC Gums, of Belcamp, Md.

Carageenan is a long chain polysaccharide widely used as a thickening and stabilizing agent, and is commonly obtained as a seaweed extract. This hydrocolloid is not only a moderate thickener for use in various foods; it has also been shown to effectively lower LDL cholesterol. A suitable source of carageenan is Lactarin DX 2015 Carageenan, available from FMC Biopolymer, of Philadelphia, Pa.

It is to be noted that some of the fibers noted above comprise a protein fraction, and that several fibers are to be included in the composition, along with their respective protein fractions in order to form a more synergistic composition, thereby improving its nutritional value. Specifically, it is known that sesame flour comprises 51% protein, soy fiber 13% protein, and flax meal 20% protein. To the group of fibers listed above, we have found it appropriate to add a specific protein component to attain the optimal results. This complex mixture of proteins and fibers combines to slow gastric emptying, and thus prolong satiety. When the digesta leaves the stomach, it moves rapidly through the intestines and colon. The protein source is generally a non-animal based protein, and may preferably be a vegetable protein, selected from the group consisting of soy, whey, caseinate, safflower, sunflower, linseed, almond, peanut, walnut, cottonseed, and sesame proteins. Egg albumin is also an alternative source of suitable protein for the present invention. The protein component of the invention is preferably provided by means of soy protein, a high quality protein currently used in both human and animal diets to great advantage, unencumbered by a high fat content so often associated with animal proteins such as meats, eggs, and dairy products. As previously noted, various forms of soy protein are available, i.e. soy flour, soy concentrate, and soy isolate. The preferred soy protein for use in the present invention is a soy isolate extracted with water and ultra filtration. Of course, the preferred fortified soy isolate protein, in combination with fewer than all of the indicated fibers will provide a benefit as well, although the maximum benefit is achieved through the use at least four of the fiber components, each of which adds a specific benefit to the mixture of components. A suitable isolated soy protein, commercially available from The Solae Company, of St. Louis, Mo., under the designation FXP HO219D, has also been shown to have cholesterol-lowering properties, which appear to be separate and distinct from its low fat content. These properties are believed to be the result of the specific amino acid building blocks, which make up soy protein, in comparison with the amino acid makeup of animal proteins. Proteins, including soy proteins, are an important component of any dietary supplement. As previously stated, a low calorie diet regimen often leads to inadvertent body protein loss, which is distinctly harmful and undesirable. Thus, a good quality protein is essential in any dietary supplement, and especially for those seeking a weight reduction. The use of protein is also well known to provide a positive effect upon serum lipids, serum lipoproteins, and plasma total homocysteine.

As previously indicated, it is not necessary to utilize all of the fibers of the group set forth above to achieve a favorable result. However, we have found that the use of less than about four members of the full group will lessen the beneficial results attained from the invention. It is also to be noted that the present invention is designed to provide the ideal balances of soluble and insoluble fibers, as well as an optimal amino acid profile to provide the greatest benefit to colon health, lowering of cholesterol, and effect upon weight maintenance of the consumer. In addition to the fibers and the soy protein source set forth above, coloring agents and flavoring agents may be added to improve palatability. For example, the addition of vanillin or artificial vanilla flavoring has been found to be very helpful, and the use of an artificial color, is beneficial. Preferred flavoring agents include N&A Vanilla 1107A, and Natural Vanilla SD-2448, both available from Ottens Flavors, Inc., of Philadelphia, Pa.; Citri Sweet, available from Roxlor International, of Wilmington, Del.; and citric acid, preferably anhydrous, readily available from multiple sources. Preferred coloring agents may be selected from any of the conventional food coloring agents known in the food industry, and include FD&C Yellow #6 Alum. Lake 35-42%, from Sensient Food Colors, of St. Louis, Mo.

Acceptable and preferred concentrations of the components of the present invention, as well as an optimal embodiment of the invention, are set forth in the table which follows.

TABLE 1

| INGREDIENT | RANGE (%) | PREFERRED (%) | OPTIMAL (%) |
|---|---|---|---|
| Isolated soy protein | 26–36 | 30–34 | 32 |
| Whey protein | 26–36 | 30–34 | 32 |
| Caseinate | 26–36 | 30–34 | 32 |
| Sunflower protein | 26–36 | 30–34 | 32 |
| Safflower protein | 26–36 | 30–34 | 32 |
| Linseed protein | 26–36 | 30–34 | 32 |
| Almond protein | 26–36 | 30–34 | 32 |
| Peanut protein | 26–36 | 30–34 | 32 |
| Walnut protein | 26–36 | 30–34 | 32 |
| Cottonseed protein | 26–36 | 30–34 | 32 |
| Sesame protein | 26–36 | 30–34 | 32 |
| Soy fiber | 8–20 | 10–15 | 12 |
| Cat fiber | 8–20 | 10–15 | 12 |
| Psyllium | 8–20 | 10–15 | 12 |
| Beet fiber | 8–18 | 10–14 | 12 |
| Indigestible dextrin | 8–18 | 10–14 | 12 |
| Flaxseed | 7–14 | 9–11 | 10 |
| Sesame flour | 4–8 | 4–6 | 5 |
| Barley flour/bran | 4–8 | 4–6 | 5 |
| Soy flour | 4–8 | 4–6 | 5 |
| Rice bran | 3–9 | 4–6 | 5 |
| Guar | 0–5 | 1–3 | 2 |
| Pectin | 0–5 | 1–3 | 2 |
| Carageenan | 0–5 | 1–3 | 2 |
| Flavoring agent | 0–3 | 0–3 | 0.25 |
| Coloring agent | 0–1 | 0–1 | 0.25 |

### EXAMPLE I

As a test formulation of the invention, a mixture of the following components was prepared:

| | |
|---|---|
| 1. Soy protein concentrate (72% Protein) | 48.0% |
| 2. Oat bran | 14.5 |
| 3. Barley bran | 9.5 |
| 4. Soy fiber | 8.0 |
| 5. Rice bran | 7.0 |
| 6. Beet fiber | 7.0 |
| 7. Psyllium fiber | 3.0 |
| 8. Guar gum | 2.0 |
| 9. Lecithin | 1.0 |

It is to be noted that the soy protein employed in this test was a concentrate, rather than an isolate (88% protein) which is the preferred form of protein, and that the mix contained no coloring or flavoring agents, but did contain lecithin, a wetting agent. This formulation was administered to a group consisting of 30 randomly selected female individuals, ranging in age from 27 to 65 years, for a period of 3 weeks. All received an orientation at the commencement of the study, which included a basic commentary on nutrition and dieting, and emphasized that an objective of the program was to demonstrate that one could lose a moderate amount of weight by staying within one's normal eating patterns, while adding a supplement prior to each meal. In the context of this study, a maximum weekly weight loss of 2 pounds was sought. The participants kept a diary of foods consumed during the study, including snacks and beverages, were weighed on a weekly basis, and refrained from any other dietary efforts to accelerate weight loss. At the end of this period, a large majority of the subjects indicated feeling comfortable, with improved overall well-being while taking the product. While weight loss varied among the test subjects, almost all registered a weight loss. At the conclusion of the study, the results of 4 persons were considered invalid for a variety of reasons. Of the remaining participants, 3.8% lost 8 pounds, 50.0% lost from 6 to 7.5 pounds, 30.8% lost from 3.5 to 5.25 pounds, and 3.8% lost 2 pounds, while 3.8% gained 1.6 pounds, 3.8% gained 1.25 pounds, and 3.8% remained the same weight. Of the 30 original participants, 28 signed up for an additional 3 week cycle, which was not monitored. Informal reports indicated similar results, with satisfaction of the participants shown by desire to purchase the product for continued use. This study validated the expectations of the inventors, and led to further refinement of the formulation to improve flavor, palatability, and overall balance of the ingredients, leading to the ranges set forth in the table above.

### EXAMPLE II

After further experimentation, a sample of the invention was prepared in accordance with the preferred formulation set forth in the table above. For this experiment, an isolated soy protein was employed, permitting the formulation to contain less of the protein, and thus more fiber. Since rice bran was found to be less efficacious, alternative fibers were employed. A coloring agent was employed to provide a more visually attractive product, and a flavoring agent was employed to mask the flavor imparted by the soy protein. In addition, a finer granular dispersion of the components was achieved, permitting the elimination of lecithin as a wetting agent. This formulation comprised the following ingredients, in the stated concentrations based upon the weight of the total mixture.

| | |
|---|---|
| Isolated Soy Protein FXP HO.219D | 32.0% |
| Soy Fiber (Fibrim 1020) | 12.0 |
| Oat Fiber (300) | 12.5 |
| Psyllium (40 mesh) | 10.0 |
| Flaxseed (BevGrad ™) | 10.0 |
| Indigestible dextrin (Fibersol 2 ™) | 10.0 |
| Sesame Flour | 5.0 |
| Guar NT 3500A | 2.0 |
| Pectin (1694) | 2.0 |
| Carageenan (Lactarin DX 2015) | 2.0 |
| Citric Acid (anhydrous) | 1.0 |
| Citri Sweet | 1.0 |
| FD&C Yellow #6 Alum. Lake 35–42% | 0.25 |
| N&A Vanilla 1107a | 0.25 |
| | 100.0 |

Upon analysis, this formulation is found to comprise (on a per serving basis) 27.8% protein, 46.2% fiber (16.5% soluble, 29.7% insoluble), and 5.8% fat (0.9% saturated, 4.9% unsaturated). The product was given to a focus group for evaluation, in 10 gram serving pouches, to be used as a supplement to each of three meals daily, over a period of 21 days. The focus group comprised 18 individuals, 10 female and 8 male, from 24 to 32 years in age. The announced objective of the study was to determine any weight or cholesterol changes after three weeks use of the supplement. Participants were urged to not change their normal eating habits, merely to add the supplement to their normal diet and to consume at least 4 glasses of water daily, not counting any other beverages consumed. The participants were asked to avoid taking any other measures to influence their weight or cholesterol count. The orientation included a full product description so that each participant knew exactly what was being consumed. Subjects were instructed to take the product once daily for the first week, preferably 15 minutes before their largest meal of the day. For the second and third weeks, they were asked to take the product twice daily, preferably one before breakfast, and the other before the largest meal of the day. The product could be consumed by stirring into a glass of water, juice, or other beverages, and the orientation included other suggestions for use, such as with yogurt or applesauce. If water was used, it was to be included in their daily allotment of four glasses. Participants took their first dose of the product during the orientation meeting. The participants were weighed, and received a Total Cholesterol reading. LDL and HDL were not determined.

The dominant reaction of the subjects was a new feeling of comfort, or well-being, in the abdomen (not the stomach). While not fully understood, it is believed that this feeling of well-being results from the product eliminating from the body, or otherwise neutralizing, substances that could cause toxicity or discomfort, a view generally supported in the extensive literature on the individual ingredients contained in the product. There were no reports of discomfort, gas, or bloating. The group also reported a higher degree of satiety than in Example I, probably resulting from the use of an isolate of protein rather than a concentrate, and from a more synergistic selection of fibers. Reactions to the most pleasant way to consume the product were almost alike, that taking the product with a beverage or food as sampled at the orientation was preferred. Specifically, 4 participants reported no strong preference for either using water or another beverage or food, while 14 preferred juice, beverage, or a food/snack item.

The individual pouch serving was mentioned as an important convenience. The taste (or lack thereof), color, particle size and dispersibility of the product were reviewed, with no changes suggested, although some suggested other product forms, such as tablets, inclusion in bars, etc.

Laxation improved for 5 participants, who reported occasional constipation, and most noted an increased frequency of bowel movements, but no loose stool or diarrhea, and no discomfort were reported. One person reported occasional “dry stool”, but did admit some carelessness in consuming a full 4 glasses of water daily. The results of total cholesterol measurements showed that 3 participants exhibited no change, 7 showed a decrease of from 5 to 7.5%, and 6 showed a decrease of from 8 to 11%. Two participants did not appear at the final meeting for weighing and cholesterol testing. In addition, 2 participants showed no weight loss, while 5 persons lost from 3 to 3.5 pounds, and 9 persons lost from 3.5 to 5 pounds. This weight loss is consistent with recommendations of not exceeding weight loss of 2 pounds per week, to avoid deprivation. Little loss, if any, was registered during the first week, reflecting that the product was added to an unchanged diet in stages, and it's effect upon satiety and appetite are not quickly noted.

The invention being thus described, it will be obvious that the invention may be varied in numerous ways. Such variations are not to be considered a departure from the spirit and scope of the present invention, and all such modifications are intended to be included within the scope of the following claims.

1. A dietary supplement consisting essentially of a uniform mixture of protein and a plurality of dietary fibers.

2. The supplement of claim 1, wherein said plurality of dietary fibers are selected from the group consisting of soy fiber, oat fiber, psyllium, sesame flour, flaxseed, barley, indigestible dextrin fiber, beet fiber, rice bran, guar gum, pectin, and carageenan.

3. The supplement of claim 2, wherein said protein is selected from the group consisting of soy, whey, caseinates, safflower, sunflower, linseed, almond, peanut, walnut, cottonseed, and sesame proteins.

4. The supplement of claim 3, wherein said dietary fiber comprises a mixture of at least 4 fibers.

5. The supplement of claim 4, wherein said mixture of dietary fiber includes soy fiber, oat fiber, psyllium, and guar gum.

6. The supplement of claim 3, wherein said protein is a soy protein isolate.

7. The supplement of claim 6, wherein said mixture dietary fibers includes soy fiber, oat fiber, psyllium, and guar gum.

8. The supplement of claim 3, further comprising up to about 3.0 percent flavoring agent, and up to about 1.0 percent coloring agent.

9. A dietary supplement consisting essentially of a uniform mixture of from about 26 to about 36 weight percent protein selected from the group consisting of soy, whey, caseinates, safflower, sunflower, linseed, almond, peanut, walnut, cottonseed, and sesame proteins, the balance comprising a plurality of fibers selected from the group consisting of soy fiber, oat fiber, psyllium, sesame flour, flaxseed, barley, indigestible dextrin fiber, beet fiber, rice bran, guar gum, pectin, and carageenan.

10. A dietary supplement as set forth in claim 9, wherein the fibers comprise from about 8 to about 20 weight percent soy fiber, from about 8 to about 20 weight percent oat fiber, from about 8 to about 20 weight percent psyllium, from about 4 to about 8 weight percent sesame flour, from about 7 to about 14 weight percent flaxseed, from about 4 to about 8 weight percent barley, from about 8 to about 18 weight percent indigestible dextrin fiber, from about 8 to about 18 weight percent beet fiber, from about 3 to about 9 weight percent rice bran, from about 0 to about 5 weight percent guar gum, from about 0 to about 5 weight percent pectin, and from about 0 to about 5 weight percent carageenan.

11. The supplement of claim 10, wherein said dietary fiber comprises a mixture of at least 4 fibers.

12. A dietary supplement as set forth in claim 11, further comprising up to about 3.0 weight percent coloring agent, and up to about 1.0 weight percent flavoring agent.

13. A dietary supplement as set forth in claim 12, wherein said protein comprises a soy protein isolate, and said fiber comprises a soy fiber, oat fiber, psyllium, and guar gum.

14. A dietary supplement as set forth in claim 10, wherein said dietary fiber comprises a mixture of from about 5 to about 8 fibers, further comprising up to about 1.0 weight percent coloring agent, and up to about 1.0 weight percent flavoring agent.

15. A dietary supplement comprising about 32 weight percent isolated soy protein, about 12 percent soy fiber, about 12.5 percent oat fiber, about 10 percent psyllium, about 10 percent flaxseed, about 10 percent indigestible dextrin, about 5 percent sesame flour, about 2 percent guar gum, about 2 percent pectin, about 2 percent carageenan, about 2 percent flavoring, and coloring agent.

16. A composition of matter comprising a uniform mixture consisting essentially of from about 26 to about 36 weight protein, from about 64 to about 74 weight percent dietary fiber comprising a plurality of fibers, and up to about 1 percent each of coloring agents and flavoring agents.

17. A composition of matter as set forth in claim 16, wherein said protein is selected from the group consisting of soy, whey, caseinates, safflower, sunflower, linseed, almond, peanut, walnut, cottonseed, and sesame proteins, and said dietary fiber comprises fibers selected from the group consisting of soy fiber, oat fiber, psyllium, sesame flour, flaxseed, barley, indigestible dextrin fiber, beet fiber, rice bran, guar gum, pectin, and carageenan.

18. A composition of matter as set forth in claim 17, wherein said protein comprises isolated soy protein, and said dietary fiber comprises a mixture of soy fiber, oat fiber, psyllium, and guar gum.

19. A composition of matter as set forth in claim 18 comprising from about 30 to about 34 weight percent isolated soy protein, from about 10 to about 15 weight percent soy fiber, from about 10 to about 15 weight percent oat fiber, from about 10 to about 15 weight percent psyllium, about 1 to about 3 weight percent guar gum, and from about 18 weight percent to about 39 weight percent additional fibers selected from the group consisting of beet fiber, indigestible dextrin, flaxseed, sesame flour, barley, rice bran, pectin, and carageenan.

20. A composition of matter as set forth in claim 16 comprising about 32 weight percent isolated soy protein, about 12 percent soy fiber, about 12.5 percent oat fiber, about 10 percent psyllium, about 10 percent flaxseed, about 10 percent indigestible dextrin, about 5 percent sesame flour, about 2 percent guar gum, about 2 percent pectin, about 2 percent carageenan, about 2 percent flavoring and coloring agent.
